(19) United States
(12) Patent Application Publication
(10) Pub. No.: US 2023/0263964 A1
(43) Pub. Date: Aug. 24, 2023

(54) Detachable Needle Syringe Having Reduced Dead Space

(71) Applicant: Becton, Dickinsonand Company, Franklin Lakes, NJ (US)

(72) Inventors: Christina D'Arrigo, Hoboken, NJ (US); Eric Schiller, Kinnelon, NJ (US)

(73) Assignee: Becton, Dickinsonand Company, Franklin Lakes, NJ (US)

(21) Appl. No.: 18/110,223

(22) Filed: Feb. 15, 2023

Related U.S. Application Data
(63) Continuation of application No. 12/376,151, filed on Feb. 3, 2009.
Application No. 12/376,151 is a continuation of application No. 11/462,083, filed on Aug. 3, 2006, now abandoned.

(51) Int. Cl. A61M 5/34

(52) U.S. Cl. CPC A61M 5/347; A61M 2005/31516

(57) ABSTRACT

A syringe and detachable needle assembly having reduced dead space features includes an elongate syringe barrel having open distal end including a collar and a distally facing annular surface projecting inwardly from the collar. A needle assembly includes a hub having a proximally facing annular surface and an outwardly projecting annular sealing ring positioned distally from the surface. The needle assembly also includes a cannula having a lumen therethrough connected to the distal end of the hub. Structure is provided for engaging the hub to the barrel resulting in the annular surface of the barrel contacting the annular surface of the hub to form a primary seal between said barrel and said hub and the annular sealing ring contacting the collar to form a secondary seal between the barrel and the hub.

### CROSS-REFERENCE TO RELATED APPLICATIONS

This application is a continuation of U.S. Pat. Application Serial No. 12/376,151, filed on Feb. 3, 2009, now pending, which is a National Stage entry of PCT/US07/17275 filed on Aug. 2, 2007, which is a continuation of U.S. Pat. Application Serial No. 11/462,083 filed Aug. 3, 2006, now abandoned, the disclosures of which are incorporated herein by reference in their entirety.

### FIELD OF THE INVENTION

The present invention relates to syringes with removable needle assemblies. More particularly, the present invention relates to a syringe and needle assembly having structure for consistently minimizing dead space in a syringe barrel while containing an effective seal between the syringe barrel and the needle assembly.

### BACKGROUND OF THE INVENTION

Syringe assemblies designed for use with replaceable needle assemblies usually contain a luer slip or locking luer type fitting for securing the needle assembly to the syringe barrel. These connections rely on an elongate frusto-conically shaped syringe barrel tip which frictionally engages a frusto-conically shaped passageway in a needle hub. The relatively small acute angle of the syringe tip, measured from the longitudinal axis of the syringe barrel, provides an excellent seal between the syringe tip and the hub. However, slight variations in the angle or diameter of the tapered surfaces of the syringe barrel and/or the needle hub produce large variations in the relative position of the needle hub with respect to the end of the barrel and, therefore, variations in dead space. The space inside the barrel tip and between the end of the barrel tip and the end of the cavity in the hub constitutes a dead space containing liquid that cannot be delivered by the syringe.

In the case of expensive medications and multi-patient immunization programs, medication lost in the dead space in single dose, multidose and prefilled syringes can become costly. This is especially true in immunization programs involving thousands of people. Controlling dead space to a minimum may result in more people being immunized with the same amount of medication provided for the program.

The prior art also teaches a flat seal, perpendicular to the longitudinal axis of the syringe barrel which mates with a flat seal on the needle hub for use with a threaded needle assembly and barrel engagement structure. The flat seal then greatly reduces the portion of the dead space attributable to variations in barrel and hub tolerances. However, imperfections on either or both of the mating flat surfaces can result in a leaky seal which allows medication to leave the syringe during use.

Although the prior art teaches many syringe barrel and needle hub connecting structures, there is still a need for a low dead space syringe which is easy to manufacture and provides consistently low dead space from syringe to syringe and an effective seal between the needle assembly and the syringe barrel.

### SUMMARY OF THE INVENTION

A syringe and detachable needle assembly having reduced dead space features includes an elongate syringe barrel having a longitudinal axis, an inside surface defining a chamber for retaining fluid, an open proximal end, an open distal end including a collar and a distally facing annular, preferably flat surface projecting inwardly from the collar. A portion of the collar includes a cylindrically shaped, axially aligned circular sidewall. A needle assembly includes a hub and a cannula. The hub includes a body portion having a proximal end, a distal end, a conduit therethrough, a proximally facing annular, preferably flat surface on the body portion contacting the annular surface of the barrel forming a primary seal between the hub and the barrel. The hub further includes an outwardly projecting annular sealing ring engaging the cylindrically shaped axially aligned portion of the sidewall of the collar forming a secondary seal between the hub and the barrel. The needle assembly further includes a cannula having a distal end, a proximal end and a lumen therethrough. The proximal end of the cannula is connected to the distal end of the hub so that the lumen of the cannula is in fluid communication with the chamber in the barrel. Structure is also provided for releasably engaging the hub to the collar.

The syringe assembly may also include an elongate plunger rod having a proximal end, a distal end and a stopper at the distal end of the plunger rod. The stopper is positioned in fluid tight engagement with the inside surface of the barrel for displacing fluid from the chamber through the cannula by relative motion of the plunger rod with respect to the barrel. The stopper may include a distally facing projection for partially occluding the conduit in the hub when the stopper is in its distal most position inside the barrel.

The outwardly projecting annular sealing ring can be any structure which is resilient enough to deflect upon installation of the needle assembly to the barrel so that the annular sealing ring deflects upon contact with the cylindrically shaped, axially aligned circular sidewall portion of the collar. The annular sealing ring can be an elastomeric o-ring preferably positioned in an annular groove in the hub. The o-ring preferably has a circularly shaped cross-section. The annular sealing ring can also be a tapered projection having a base adjacent to the body portion of the hub and a free end wherein the tapered projection is wider at its base than at its free end. The annular sealing ring may also be a radially outwardly projecting cantilever shaped projection. This projection may be positioned between two annular grooves on the hub. Also, the annular sealing ring may be integrally formed with the body portion of the hub.

An elongate hollow needle shield having a distal end and an open proximal end can be removably engaged to the hub so that the needle shield covers the cannula.

A needle assembly may include a hub and cannula that are integrally formed with thermoplastic material. The cannula may also be formed of metal such as stainless steel and mechanically connected to the hub. The cannula can have a sharp distal tip or a blunt distal tip. The structure for releasably engaging the hub to the collar may include at least one thread on the collar or on the hub. The at least one thread may be right-handed thread and/or a multiple lead thread.

The structure for releasably engaging the hub to the collar includes at least one thread engaging projection on the hub or the collar and at least one thread on the other of the hub or the collar.

### BRIEF DESCRIPTION OF THE DRAWINGS

FIG. 1 is an exploded view illustrating a prior art syringe and needle assembly.

FIG. 2 is a partial cross-sectional view of the prior art needle assembly of FIG. 1 connected to the prior art syringe of FIG. 1.

FIG. 3 is a prospective view of the syringe and detachable needle assembly of the present invention.

FIG. 4 is an exploded view of the syringe and detachable needle assembly of the present invention.

FIG. 5 is a side elevational view of the needle assembly, with needle shield attached, of the present invention.

FIG. 6 is a cross-sectional view of the needle assembly of FIG. 5 taken along lines 6-6.

FIG. 7 is a partial enlarged cross-sectional view of the distal end of the barrel of FIG. 4.

FIG. 8 is a partial cross-sectional view illustrating the needle assembly connected to the syringe barrel.

FIG. 9 is an alternative embodiment of the needle assembly of the present invention having an integrally formed sealing ring.

FIG. 10 is a side elevational view of another embodiment of the needle assembly of present invention.

FIG. 11 is an enlarged cross-sectional view of the distal end of the barrel for use with a needle assembly of FIG. 10.

FIG. 12 is a partial cross-sectional view illustrating the needle assembly of FIG. 10 connected to the barrel of FIG. 11.

### DETAILED DESCRIPTION

While this invention is satisfied by embodiments in many different forms, there are shown in the drawings and will herein be described in detail, preferred embodiments of the invention with the understanding that the present disclosure is to be considered exemplary of the principles of the invention and not intended to limit the invention to the embodiments illustrated. The scope of the invention will be measured by the appended claims and their equivalents.

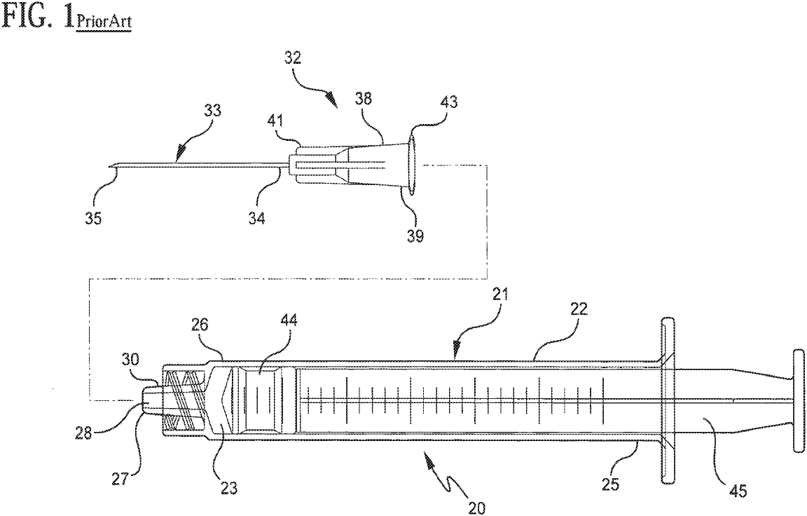

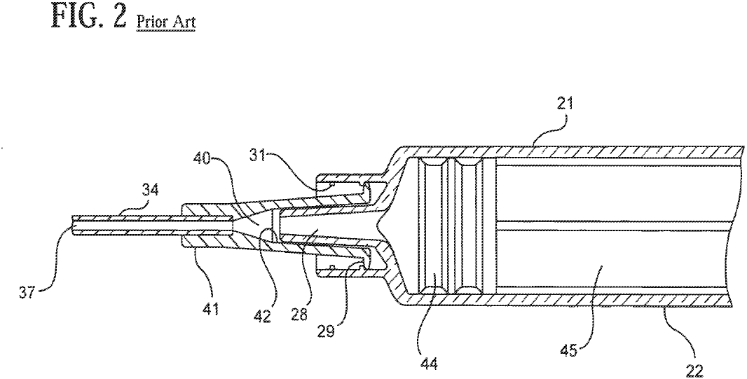

Referring to FIGS. 1-2, a prior art syringe assembly 20 includes a barrel 21 having an elongate body 22 defining a chamber 23 for retaining fluid. The barrel includes an open proximal end 25 and distal end 26 including a tip 27 having a passageway 28 therethrough in fluid communication with the chamber. The distal end of the barrel may also include a locking luer-type collar 29 concentrically surrounding tip 27. The luer collar has an internal thread 31.

A prior art needle assembly 32 includes a cannula 33 having a proximal end 34, a distal end 35 and lumen 37 therethrough. A hub 38 includes an open proximal end 39 containing a cavity 40 and a distal end 41 attached to proximal end 34 of the cannula so that the lumen of the cannula is in fluid communication with the cavity in the hub. The needle assembly is removably attached to the syringe through frictional engagement of the interior surface of the cavity in the hub and the exterior surface of tip 27. The hub also includes radial projections 43 on its proximal end for engaging thread 31 to hold the needle assembly securely to the barrel. The prior art syringe assembly further includes a resilient stopper 44 connected to an elongate plunger rod 45.

Liquid in a syringe barrel can experience very high pressures during the injection process. This is especially true in the case of high viscosity medications and/or needle cannula having a lower diameter lumen. To seal the interface between the needle hub and the syringe against these high pressures, a frusto-conically shaped engagement surface 30 on tip 27 frictionally engages a frusto-conically shaped engagement surface 42 in hub cavity 40. When the diameter of the engagement surface 42 is on the small end of its tolerance range and the diameter of the engagement surface 30 of the barrel tip is on the large end of its tolerance range, it can be seen that the barrel tip will not move as far into the cavity of the needle hub thus leaving more empty space in the hub cavity. Although the seal created by the frusto-conically shaped engagement surfaces is very effective, the components must be designed to accept all anticipated tolerance variations of the barrel tip and hub cavity so that the distance the barrel engages the hub cavity will vary throughout the tolerance range thus varying the amount of unoccluded space in the hub cavity. As can be seen in FIG. 2, the dead space includes the unoccluded space in the tip passageway 28, inherent in the luer slip design, and the unoccluded space in the hub cavity 40, partially necessitated by tolerance variations. The unoccluded space in the barrel tip is inherent in the design while the occluded space in the hub cavity will vary widely to the tolerance variation properties of the luer-type engagement. Accordingly, dead space is difficult to control in the prior art syringe using luer-type fittings.

Other prior art syringe assemblies have attempted to minimize and reduce the variability of dead space by providing frusto-conically shaped engagement surfaces which are at much larger angles with respect to the longitudinal axis of the barrel to shorten the variation in the dead space based on tolerances. The best control of dead spaces can be achieved with a flat engagement surface positioned approximately at an angle 90° with respect to the longitudinal axis of the barrel. This configuration best controls dead space caused by tolerance variation, and can overcome inherent dead space in the luer slip barrel tip. However, imperfections on the mating surfaces or variations in the flatness of these surfaces can result in a seal between the hub and the barrel which may leak initially or over a period of time. Also, the quality of the seal is directly related to the amount of force applied to the hub while engaging it to the barrel.

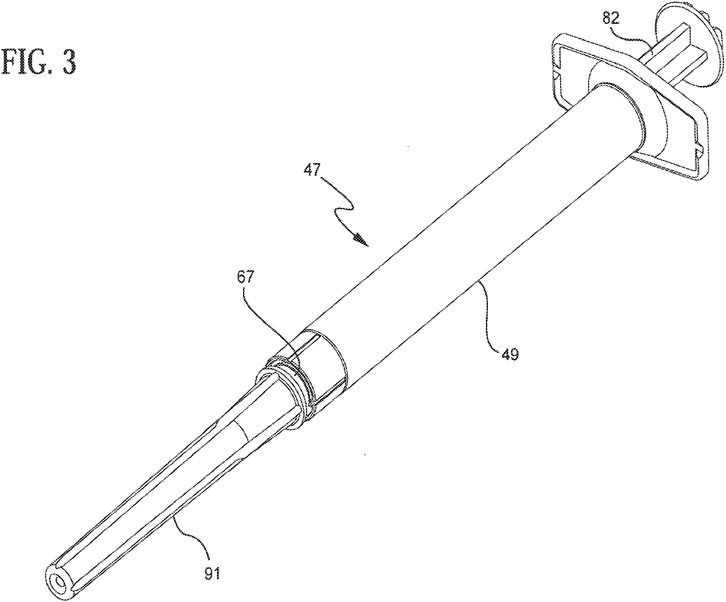

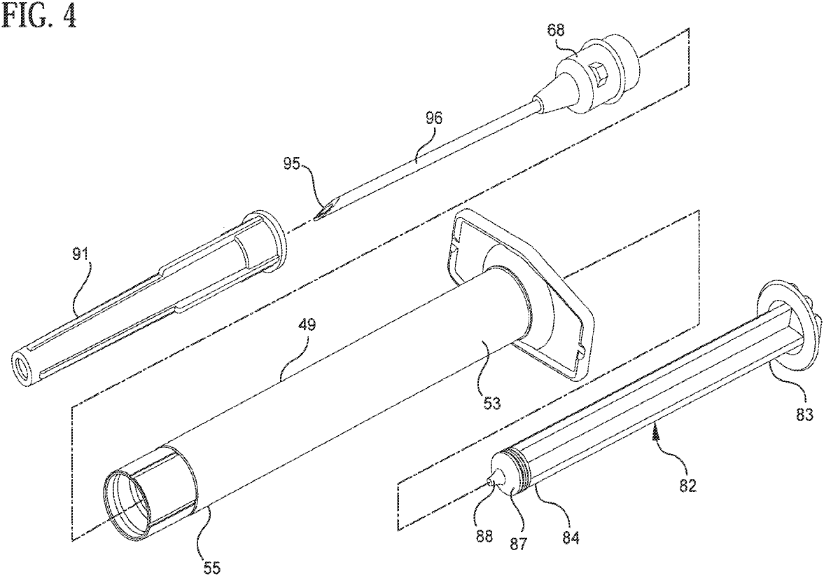

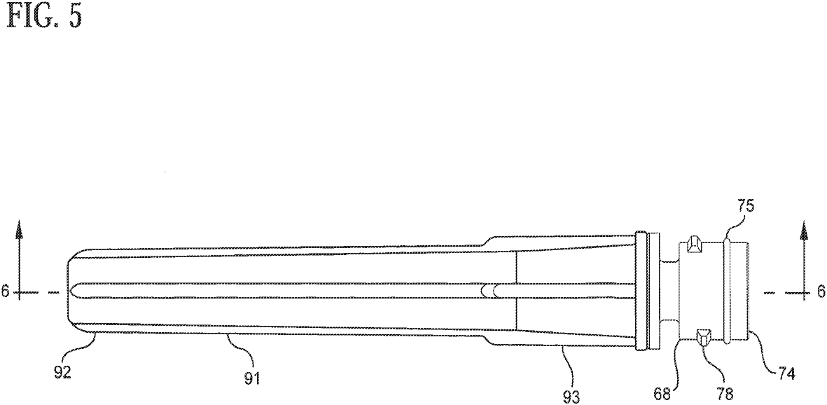

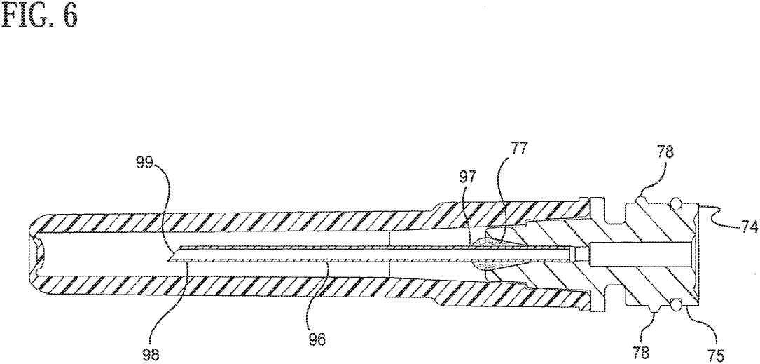

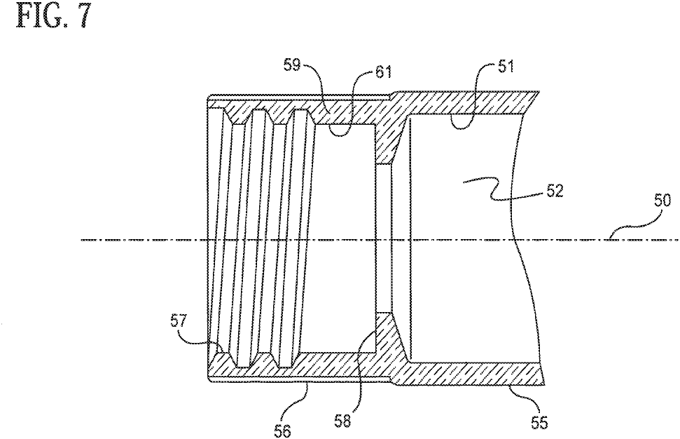

Referring to FIGS. 3-8, a syringe and detachable needle assembly 47 of the present invention, having reduced dead space features includes an elongate syringe barrel 49 having a longitudinal axis 50, an inside surface 51 defining a chamber 52 for retaining fluid. Barrel 49 further includes an open proximal end 53, an open distal end 55 including a collar 56 having a needle hub engaging structure and a distally facing annular surface 58 projecting inwardly from the collar. Annular surface 58 is preferably flat. A portion of the collar includes a cylindrically shaped, axially aligned circular sidewall.

A needle assembly 67 includes a hub 68 having a body portion 69 including a proximal end 70, a distal end 71 and a conduit 73 therethrough. A proximally facing annular surface 74 on the body portion contacts the annular surface on the barrel forming a primary seal between the hub and the barrel. Annular surface 74 is preferably flat. The hub also includes an outwardly projecting annular sealing ring 75 sealingly engaging the circular sidewall of the collar forming a secondary seal between the hub and the barrel.

In this embodiment, the annular sealing ring is an o-ring contained in annular groove 76 around the body portion of the hub. As will be more fully explained hereinafter, the outwardly projecting annular sealing ring can be any structure which is resilient enough to deflect upon installation of the needle assembly to the barrel so that the annular sealing ring deflects upon contact with circular sidewall portion of the collar. These structures are all within the purview of the present invention. The o-ring and other structures for the annular sealing ring taught herein are merely representative of these many possibilities.

Needle assembly further includes a cannula 96 having proximal end 97, a distal end 98 preferably having a sharp distal tip 95 and a lumen 99 therethrough. The proximal end of the cannula is connected to the distal end of the hub so that the lumen is in fluid communication with the chamber in the barrel. The cannula may be integrally formed with the hub such as by injection molding using thermoplastic materials or separately formed, as in this embodiment, and attached to the hub. In this embodiment adhesive 77 is used to attach the cannula to the hub, and the cannula is made of metal, preferably stainless steel.

Structure for releasably engaging the hub to the collar including hub engaging structure on the collar and complimentary collar engaging structure on the hub is provided. In this embodiment structure for releasably engaging the hub and collar includes at least one thread 57 on an inside surface 61 of the circular shaped sidewall 59. The thread may be either right-handed or left-handed, and it also may be a multiple-lead thread. The hub includes at least one thread engaging the projection. In this embodiment, there are two thread engaging projections 78, preferably on opposite sides of the hub.

The syringe assembly may also include an elongate plunger rod 82 having a proximal end 83, a distal end 84 and a stopper 87 at the distal end of the plunger rod. A stopper is slidably positioned in fluid tight engagement with the inside surface of the barrel for displacing fluid from the chamber through the cannula by relative motion of the plunger with respect to the barrel. The stopper desirably includes a distally facing projection 88 for partially occluding the conduit in the hub when the stopper is in its distal most position inside the barrel to further reduce dead space.

The needle assembly may further include an elongate, hollow needle shield 91 having a distal end 92 and open proximal end 93 removably engaged to the hub so that the needle shield covers the cannula.

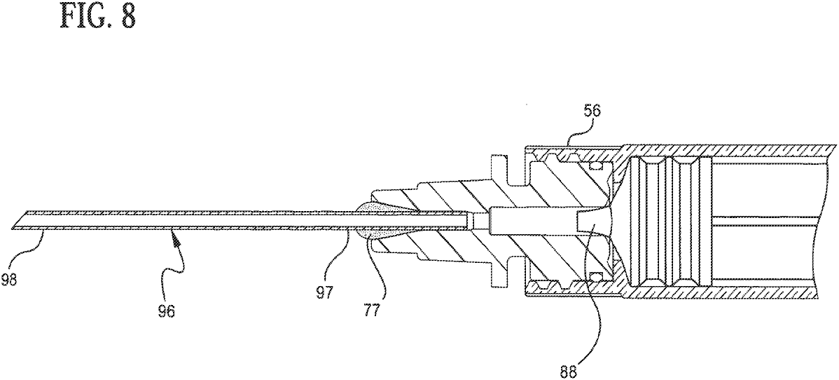

The needle assembly maybe installed or removed from the barrel by grasping the needle shield and rotating the hub in the appropriate direction to install or disengage the needle assembly. During installation, rotation of the hub will cause the needle engaging projections of the hub to follow the thread until proximally facing annular surface 74 of the hub contacts distally facing annular surface 58 of the barrel to form primary seal between the hub and the barrel. In this position, outwardly projecting annular sealing ring 75 is deflected as illustrated in FIG. 8 to form a secondary seal between the hub and the barrel. The secondary seal is a backup seal in the event that there are imperfections or other discontinuities on the annular surfaces or other factors that compromise their sealing ability or in the event that the user fails to provide enough rotational torque while connecting the hub to the barrel to adequately engage the primary seal.

It should be noted that the proximally facing annular surface 74 when it is flat on the hub and the distally facing annular surface 58 when it is flat on the barrel need not be aligned exactly at 90° from the longitudinal axis. A broad range of angles will work with the angles of 88° to 92° being desired and 90° being preferred.

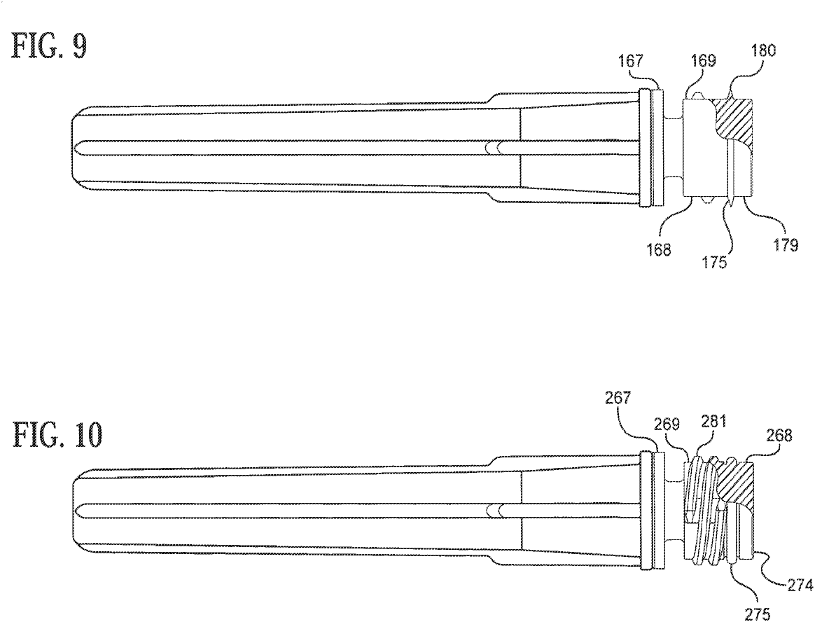

FIG. 9 illustrates an alternative embodiment of the needle assembly of the present invention. In this embodiment, needle assembly 167 performs similarly to the needle assembly of FIGS. 3-8. In this embodiment, hub 168 has an outwardly projecting annular sealing ring 175 which is integrally molded with body portion 169 of the hub. The annular sealing ring is a tapered projection having a base 179 adjacent to the body portion and a free end 180. Tapered projection in this embodiment is wider at its base than at its free end. Projection functions similarly to the projection in the embodiment of FIGS. 3-8.

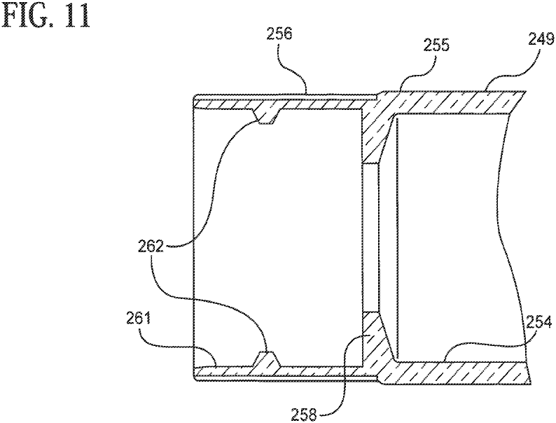

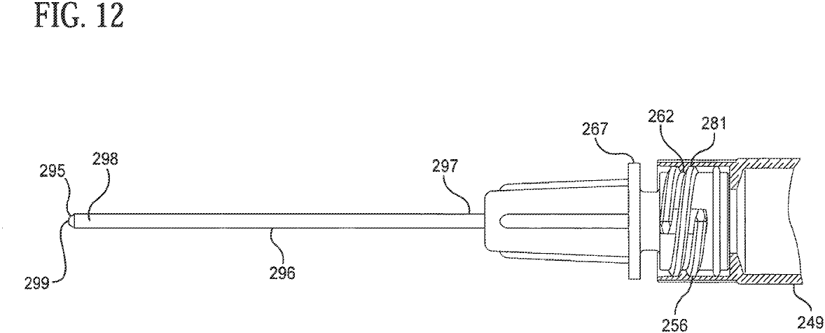

FIGS. 10,11 and 12 illustrate another alternative embodiment of the syringe assembly of the present invention. In this embodiment, needle assembly 267 includes a hub 268 having a body portion 269 wherein the body portion includes at least one thread 281, an outwardly projecting cantilever shaped annular sealing ring 275 positioned between annular grooves 276 and a proximally facing annular flat surface 274. Needle assembly 267 further includes a cannula 296 having a proximal end 297, a distal end 298, preferably having a blunt distal tip 295 and a lumen 299 therethrough. The proximal end of the cannula is connected to the distal end of the hub so that the lumen is in fluid communication with the chamber and the barrel. In this embodiment, the cannula is integrally formed with the hub using thermoplastic materials and an injection molding process. A blunt tip is well suited for use with fluid transfer devices having a split septum and other fluid transfer devices designed for accepting blunt cannula.

An elongate barrel 249 includes an inside surface 251 defining a chamber 252 and an open distal end 255 and a collar 256. The collar includes an inside surface 261 having at least one thread engaging projection. In this embodiment there are two thread engaging projections 262 facing inwardly for engaging thread 281 on hub 268. The components of this embodiment function similarly to the embodiment of FIGS. 3-8. In this embodiment, proximally facing annular flat surface 274 on the hub engages distally facing annular flat surface 258 in the barrel to form the primary seal between the hub and the barrel. Cantilever shaped outwardly facing annular sealing ring 275 will be deflected by inside surface 261 during installation to provide a secondary seal between the hub and the barrel.

There are many ways to configure the hub and the collar of the barrel including a hub having an additional skirt which surrounds the collar of the syringe barrel. With this construction, the outside of the collar may include at least one thread or at least one thread-engaging projection while the inside of the hub skirt will include the other of a thread or at least one thread-engaging projection. The engagement between the hub and the collar, in all configurations, can include a thread or thread-like structure on the hub and a thread or thread-like structure on the collar. All of these structures are within the purview of the present invention and the embodiments taught hereinabove are merely representative of these many possibilities.

What is claimed is:

1. A syringe and detachable needle assembly having reduced dead space features comprising:
an elongate syringe barrel having a longitudinal axis, an inside surface defining a chamber for retaining fluid, an open proximal end, an open distal end including a collar and a distally facing annular surface projecting inwardly from said collar, a portion of said collar having a cylindrically shaped, axially aligned circular side wall;
a needle assembly including a hub having a body portion including a proximal end, a distal end, a conduit therethrough, a proximally facing annular surface on said body portion contacting said annular surface of said barrel forming a primary seal between said hub and said barrel, said hub further including an outwardly projecting annular sealing ring sealingly engaging said circular side wall of said collar forming a secondary seal between said hub and said barrel, said needle assembly further including a cannula having a distal end, a proximal end and a lumen therethrough, said proximal end of said cannula being connected to said distal end of said hub so that said lumen is in fluid communication with said chamber;
wherein distally facing annular surface of the barrel is flat and proximally facing annular surface of the hub is flat;
two thread engaging projections on opposite sides of the hub;
wherein during installation, rotation of the hub will cause the needle engaging projections of the hub to follow the thread until the proximally facing annular surface of the hub contacts distally facing annular surface of the barrel to form primary seal between the hub and the barrel;
the outwardly projecting annular sealing ring is positioned between the threaded projections and the proximal facing annular surface of the hub;
means for releasably engaging said hub to said collar including at least one thread on an inside surface of said collar or at least one thread on an outside surface of said hub and the needle assembly is installed or removed from the barrel by rotating the hub, and wherein the secondary seal is a backup seal in the event that the user fails to provide enough rotational torque while connecting the hub to the barrel to adequately engage the primary seal.

2. The syringe assembly of claim 1, further including an elongate plunger rod having a proximal end, a distal end and a stopper at said distal end of said plunger rod, said stopper being slidably positioned in fluid tight engagement with said inside surface of said barrel for displacing fluid from said chamber through said cannula by relative motion of said plunger rod with respect to said barrel.

3. The syringe assembly of claim 2, wherein said stopper includes a distally facing projection for partially occluding said conduit in said hub when said stopper is in its distal most position inside said barrel.

4. The syringe assembly of claim 1, wherein said annular sealing ring is an elastomeric o-ring and wherein said o-ring has preferably a circularly shaped cross-section.

5. The syringe assembly of claim 1, wherein said annular sealing ring is a tapered projection having a base adjacent to said body portion and a free end, said tapered projection being wider at said base than at said free end.

6. The syringe assembly of claim 1, wherein said annular sealing ring is a radial, cantilever shaped projection or wherein said annular sealing ring is positioned between two annular grooves or wherein said annular sealing ring is integrally formed with said body portion.

7. The syringe assembly of claim 1, further including an elongate hollow needle shield having a distal end and an open proximal end removably engaged to said hub so that said needle shield covers said cannula.

8. The syringe assembly of claim 1, wherein said hub and said cannula are integrally formed of thermoplastic material.

9. The syringe assembly of claim 1, wherein said cannula includes a blunt distal tip or wherein said cannula is formed of metal.

10. The syringe assembly of claim 1, wherein said means for releasably engaging said hub to said collar includes at least one thread on an inside surface of said collar.

11. The syringe assembly of claim 10, wherein said at least one thread is a right-hand thread and/ or wherein said at least one thread is a multiple lead thread.

12. The syringe assembly of claim 1, wherein said means for releasably engaging said hub to said collar includes at least one thread engaging projection on said hub or at least one thread on an outside surface of said hub or at least one thread engaging projection on an inside surface of said collar.

13. A syringe and detachable needle assembly of claim 1, further comprising:
an elongate hollow needle shield having a distal end and an open proximal end removably engaged to said hub so that said needle shield covers said cannula; and
an elongate plunger rod having a proximal end, a distal end and a stopper at said distal end of said plunger rod, said stopper being slidably positioned in fluid tight engagement with said inside surface of said barrel for displacing fluid from said chamber through said cannula by relative motion of said plunger rod with respect to said barrel.
